(19) United States
(12) Patent Application Publication
(10) Pub. No.: US 2024/0252147 A1
(43) Pub. Date: Aug. 1, 2024

(54) METHODS AND SYSTEMS FOR CAPACITIVE MICROMACHINED ULTRASONIC TRANSDUCERS

(71) Applicant: GE Precision Healthcare LLC, Wauwatosa, WI (US)

(72) Inventor: Rupak Bardhan Roy, Nice (FR)

(21) Appl. No.: 18/163,209

(22) Filed: Feb. 1, 2023

(51) Int. Cl. A61B 8/00

(52) U.S. Cl. CPC A61B 8/4483

(57) ABSTRACT

Various methods and systems are provided for a microelectromechanical systems (MEMS) device. In one example, the MEMS device may include a silicon wafer with a first oxide coating, a cavity in the first oxide coating having a substantially flat floor formed of a layer of the first oxide coating and etched to a first depth prior to deposition of a masking layer, and a membrane coupled to the silicon wafer and spaced away from the substantially flat floor of the cavity by a second depth of the cavity. The second depth is a sum of the first depth and a height of a second oxide coating formed at the post regions of the MEMS device after the masking layer is removed from the post regions.

### FIELD

Embodiments of the subject matter disclosed herein relates to microelectromechanical systems (MEMS) devices and, in particular, micromachined ultrasonic transducers.

### BACKGROUND

A microelectromechanical systems (MEMS) ultrasound device (hereafter, MEMS device) may be used for imaging and/or therapy targets such as organs and soft tissues in a human body, as well non-human targets. For example, the MEMS device may be used for applications such as ultrasound/acoustic sensing, non-destructive evaluation (NDE), ultrasound therapy (e.g., High Intensity Focused Ultrasound (HIFU)), etc., in addition to ultrasound imaging of humans, animals, etc. The MEMS device may rely on vibration of a membrane with a first electrode to receive and transmit signals. The membrane may be spaced away from a second electrode by a gap in the MEMS device. Thus, a presence of protruding structures within the gap may interfere with vibration of the membrane and degrade a performance of the MEMS device.

MEMS devices may use real time, non-invasive high frequency (e.g., in a range of 100 KHz to tens of MHz) sound waves to produce a series of two-dimensional (2D) and/or three-dimensional (3D) images. The sound waves may be transmitted by a transmit transducer, and the reflections of the transmitted sound waves may be received by a receive transducer. The received sound waves may then be processed to display an image of the target. For some types of MEMS devices used as a transmit transducer and/or a receive transducer, such as a capacitive micromachined ultrasound transducer (CMUT), the CMUT may include a top electrode and a bottom electrode. The top electrode may move upon receiving electrical signals to generate sound waves or may move upon receiving sound waves to generate electrical signals that can be processed. The top electrode and the bottom electrode may be separated by a gap, where the gap may comprise some level of vacuum or filled with, for example, air. However, processes for fabricating CMUTs may generate structures within the gap which may adversely affect a performance of the CMUTs.

### BRIEF DESCRIPTION

In one embodiment, a microelectromechanical systems (MEMS) device comprises a silicon wafer with a first oxide coating and a cavity in the first oxide coating having a substantially flat floor formed of a layer of the first oxide coating that is continuous with the first oxide coating at post regions of the MEMS device, the cavity etched prior to deposition of a masking layer. The MEMS device further includes a membrane coupled to the silicon wafer and spaced away from the substantially flat floor of the cavity by a depth of the cavity, wherein the depth of the cavity is a sum of a height of the cavity prior to deposition of the masking layer and a height of a second oxide coating formed at the post regions of the MEMS device after the masking layer is removed from the post regions. As an example, the substantially flat floor may have a root means square (RMS) roughness value of less than 0.1 nm. In this way, the MEMs device may be fabricated in a low cost manner that precludes formation of undesirable structures in the cavity.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows a block diagram of an exemplary ultrasound system that may be used in ultrasound imaging, in accordance with various embodiments;

FIGS. 2A-2F show an example of a first process for treating a wafer for a MEMS device, in accordance with various embodiments;

FIGS. 3A-3G show an example of a second process for treating a wafer for a MEMS device, in accordance with various embodiments;

FIGS. 4A-4C show an example of a third process for fabricating a CMUT using a wafer treated according to the first process of FIGS. 2A-2F or the second process of FIGS. 3A-3F, in accordance with various embodiments;

FIG. 5 shows an example of a method for fabricating a MEMS device.

FIGS. 6A-6C shows alternate steps for the first process of FIGS. 2A-2F.

FIG. 7 shows an example of an undesirable bird-beak structure in a cavity floor of a CMUT.

### DETAILED DESCRIPTION

The embodiments disclosed herein may provide microelectromechanical systems (MEMS) devices, such as micromachined ultrasonic transducers (CMUTs), without undesirable structures and indentations. While a CMUT can be used for medical imaging, the CMUT may also be used for various other purposes such as, for example, ultrasound/acoustic sensing, non-destructive evaluation (NDE), ultrasound therapy (e.g., High Intensity Focused Ultrasound (HIFU)), etc., in addition to ultrasound imaging of humans or animals.

As used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, MGD, and/or sub-modes of B-mode and/or CF such as Shear Wave Elasticity Imaging (SWEI), TVI, Angio, B-flow, BMI, BMI_Angio, and in some cases also MM, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

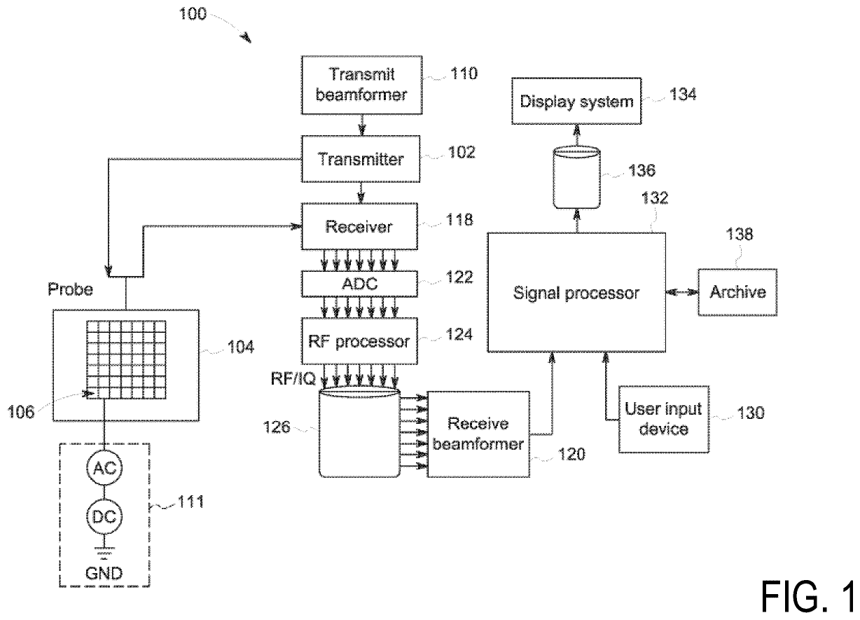

FIG. 1 is a block diagram of an exemplary ultrasound system that may be used in ultrasound imaging, in accordance with various embodiments. Referring to FIG. 1, there is shown a block diagram of an exemplary ultrasound system 100. The ultrasound system 100 comprises a transmitter 102, an ultrasound probe 104, a transmit beamformer 110, a receiver 118, a receive beamformer 120, A/D converters 122, an RF processor 124, an RF/IQ buffer 126, a user input device 130, a signal processor 132, an image buffer 136, a display system 134, and an archive 138. The circuit 111 is a non-limiting example of bias of a CMUT and variations in a configuration of the circuit 111 are possible without departing from the scope of the present disclosure.

The transmitter 102 may comprise suitable logic, circuitry, interfaces and/or code that may be operable to drive the ultrasound probe 104. The ultrasound probe 104 may comprise, for example, a single element CMUT, a 1D array of CMUTs, 2D array of CMUTs, an annular (ring) array of CMUTs, etc. Accordingly, the ultrasound probe 104 may comprise a group of transducer elements 106 that may be, for example, CMUTs. In certain embodiments, the ultrasound probe 104 may be operable to acquire ultrasound image data covering, for example, at least a substantial portion of an anatomy, such as the heart, a blood vessel, or any suitable anatomical structure. Each of the transducer elements 106 may be referred to as a channel.

The transmit beamformer 110 may comprise suitable logic, circuitry, interfaces and/or code that may be operable to control the transmitter 102 that drives the group of transducer elements I06 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes can then be received by the transducer elements 106. For example, one or more drive circuits 111 may be coupled to and drive or control the electrodes of each transducer element 106. For example, the one or more drive circuits may be coupled to separate AC and DC voltage sources.

The group of transducer elements 106 in the ultrasound probe 104 may be operable to convert the received echoes into analog signals and communicated to a receiver 118. The receiver 118 may comprise suitable logic, circuitry, interfaces and/or code that may be operable to receive the signals from the ultrasound probe 104. The analog signals may be communicated to one or more of the plurality of A/D converters 122.

Accordingly, the ultrasound system 100 may multiplex such that ultrasonic transmit signals are transmitted during certain time periods and echoes of those ultrasonic signals are received during other time periods. Although not shown explicitly, various embodiments of the disclosure may allow simultaneous transmission of ultrasonic signals and reception of echoes from those signals. In such cases, the probe may comprise transmit transducer elements and receive transducer elements.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF data, which may be, for example, I/Q signal data, real valued RF data, etc., may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

Accordingly, various embodiments may have, for example, the RF processor 124 process real valued RF data, or any other equivalent representation of the data, with an appropriate RF buffer 126. The receive beamformer 120 may comprise suitable logic, circuitry, interfaces and/or code that may be operable to perform digital beamforming processing to sum, for example, delayed, phase shifted, and/or weighted channel signals received from the RF processor 124 via the RF/IQ buffer 126 and output a beam summed signal. The delayed, phase shifted, and/or weighted channel data may be summed to form a scan line output from the receive beamformer 120, where the scan line may be, for example, complex valued or non-complex valued. The specific delay for a channel may be provided, for example, by the RF processor 124 or any other processor configured to perform the task. The delayed, phase shifted, and/or weighted channel data may be referred to as delay aligned channel data.

The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 may comprise a plurality of receive beamformers 120.

The user input device 130 may be utilized to input patient data, scan parameters, settings, select protocols and/or templates, and the like. In an exemplary embodiment, the user input device 130 may be operable to configure, manage, and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include switch(es), button(s), rotary encoder(s), a touchscreen, motion tracking, voice recognition, a mouse device, keyboard, camera, and/or any other device capable of receiving a user directive. In certain embodiments, one or more of the user input devices 130 may be integrated into other components, such as the display system 134 or the ultrasound probe 104, for example. As an example, user input device 130 may comprise a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in a live or off-line operation. In various embodiments, the processed image data can be presented at the display system 134 and/or stored at the archive 138. The archive 138 may be a local archive, a Picture Archiving and Communication System (PACS), or any suitable device for storing images and related information.

The signal processor 132 may comprise one or more central processing units, microprocessors, microcontrollers, and/or the like. The signal processor 132 may be an integrated component, or may be distributed across various locations, for example. In an exemplary embodiment, the signal processor 132 may be capable of receiving input information from the user input device 130 and/or the archive 138, generating an output displayable by the display system 134, and manipulating the output in response to input information from the user input device 130, among other things. The signal processor 132 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates may range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes worth of frames of ultrasound scan data. The frames of ultrasound scan data are stored in a manner to facilitate retrieval thereof according to its order or time of acquisition. The image buffer 136 may be embodied as any known data storage medium.

The display system 134 may be any device capable of communicating visual information to a user. For example, a display system 134 may include a liquid crystal display, a light emitting diode display, and/or any suitable display or displays. The display system 134 can be operable to present ultrasound images and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display. Additionally, while the ultrasound system 100 was described to comprise one receive beamformer 120, one RF processor 124, and one signal processor 132, various embodiments of the disclosure may use various number of processors. For example, various devices that execute code may be referred to generally as processors. Various embodiments may refer to each of these devices, including each of the RF processor 124 and the signal processor 132, as a processor. Furthermore, there may be other processors to additionally perform the tasks described as being performed by these devices, including the receive beamformer 120, the RF processor 124, and the signal processor 132, and all of these processors may be referred to as a “processor” for ease of description.

As described above, a probe for an imaging system, such as an ultrasound imaging system, or another type of medical system relying on data acquisition via acoustic signals, may incorporate CMUTs for transmitting and receiving signals. A performance of the CMUTs may rely on vibration of a membrane positioned over a gap or cavity of a CMUT. During fabrication of the CMUTs, control over dimensions and a geometry of the cavity may be challenging and may lead to formation of structures therein that may interfere with membrane vibration and lead to mechanical and electrical degradation of the CMUT.

For example, a conventional process for fabricating a MEMS device may include applying a masking layer to an area of a silicon wafer at which formation of the cavity is desired. The masking layer may be formed of a material that is resistant to oxidation and/or thermal degradation and may be used to shield, e.g., cover or mask, an area of the silicon wafer, thereby inhibiting oxidation of the silicon wafer at the area masked by the masking layer. The silicon wafer may be locally oxidized to form silicon dioxide at a surface layer of the silicon wafer. During local oxidation, the silicon oxide may diffuse laterally and seep under the masking layer. The lateral diffusion may displace edges of the masking layer upwards. In other words, seeping of the silicon dioxide may push up the edges of the masking layer, resulting in a non-planar, and non-uniform cavity floor after the masking layer is removed. For example, structures protruding upwards from the cavity floor, hereafter referred to as bird-beak structures, may remain after local oxidation is performed and the masking layer removed.

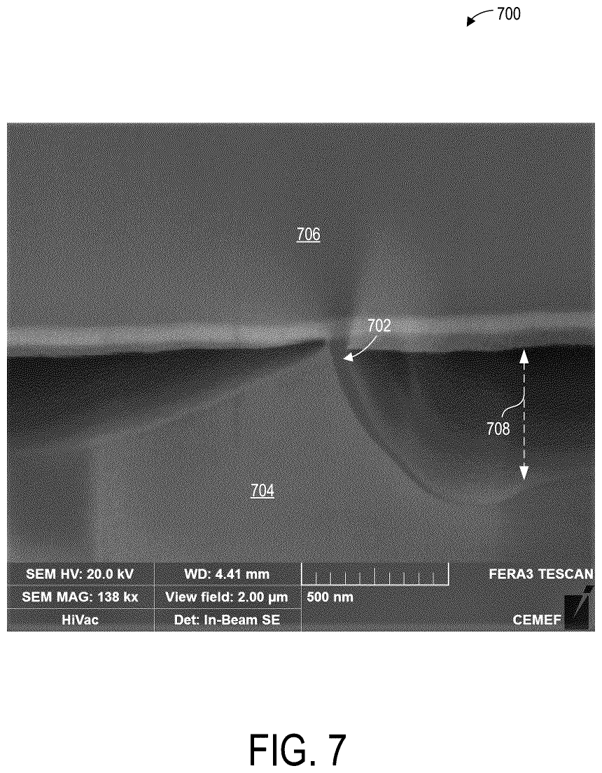

An example of a bird-beak structure 702 is shown in FIG. 7 in a SEM image 700 of a portion of a CMUT. The bird-beak structure 702 is a protruding region of a cavity floor 704 of the CMUT that is spaced away from a membrane 706 of the CMUT by a gap 708. The bird-beak structure 702, however, extends across the gap 708 and contacts the membrane 706 at least at a tip of the bird-beak structure 702. Such contact may interfere with vibration of the membrane 706 during operation of a probe incorporating the CMUT.

Attempts to address formation of undesirable bird-beak structures in cavities of MEMS devices include conducting an additional etching or polishing step after the masking layer is removed to eliminate the bird-beak structures. The additional etching/polishing step, however, may remove the silicon dioxide layer coating the cavity floor, and increase a roughness of the exposed silicon wafer at the cavity floor. The increased roughness may exacerbate a likelihood of electrical charging of the MEMS device and degrade its reliability.

In one example, the issues described above may be at least partially addressed by providing a MEMS device having a cavity in a first oxide coating of a silicon wafer, the cavity having a substantially flat floor formed of a layer of the first oxide coating. The layer of the first oxide coating of the cavity floor may be continuous with the first oxide coating at post regions of the MEMS device, where the post regions are portions of the first oxide coating bordering the cavity and forming walls of the cavity. The cavity may be etched into the first oxide coating of the silicon wafer before a masking layer is deposited, the cavity having an initial depth that is equal to or less than a thickness of the masking layer. As such, the silicon wafer may undergo a first oxidation process prior to etching of the cavity to form the first oxide coating.

The MEMS device may undergo local oxidation after the masking layer is removed from the post regions, causing a height of the post regions to increase due to formation of a second oxide coating over the first oxide coating. Upon removing the masking layer from the cavity, a final depth of the cavity may be equal to the initial depth plus a height of the post regions, the height of the post regions including both the first oxide coating and the second oxide coating.

As described above, the silicon wafer of the MEMS device may be oxidized to form the first oxide layer as a continuous outer layer of the silicon wafer before the cavity is etched. In contrast, conventional processes for fabricating MEMS devices may include depositing the masking layer prior to local oxidation to form the first oxide layer as well as to enable formation of the cavity during the local oxidation. In the conventional process, the cavity may be defined by an area covered by the masking layer and the post regions may be formed during the local oxidation, around the masking layer, which may lead to formation of a cavity with a non-uniform depth and non-planar floor. For example, the first oxide coating may diffuse laterally under the masking layer, altering a geometry of the cavity surfaces. By instead etching the cavity into an already oxidized silicon wafer, as described herein, seepage of the first oxide layer into the cavity is mitigated or slowed down, allowing the depth and a shape of the cavity to be controlled substantially.

Furthermore, by controlling the shape of the cavity to provide a cavity free of bird-beak structures, a smoothness of the cavity floor may be maintained within a desirable roughness. For example, a RMS measure of roughness of 0.1 nm or less at the cavity floor may be achieved. The resulting MEMS device, such as a CMUT, may have an increased mechanical integrity and performance relative to a CMUT fabricated via the conventional LOCOS process, while maintaining costs low and prolonging a useful life of the CMUT.

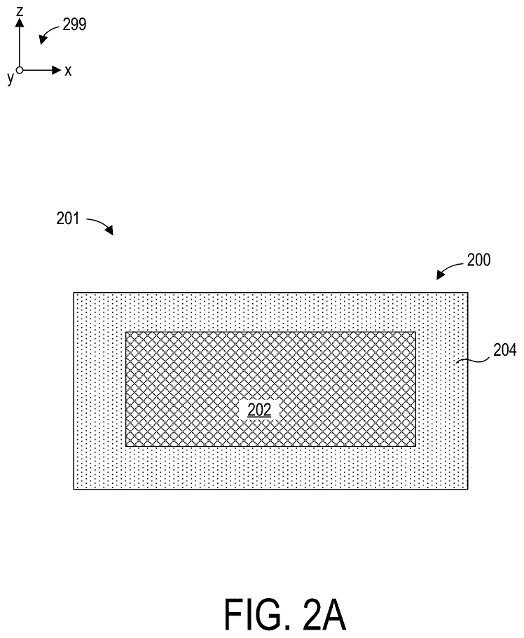

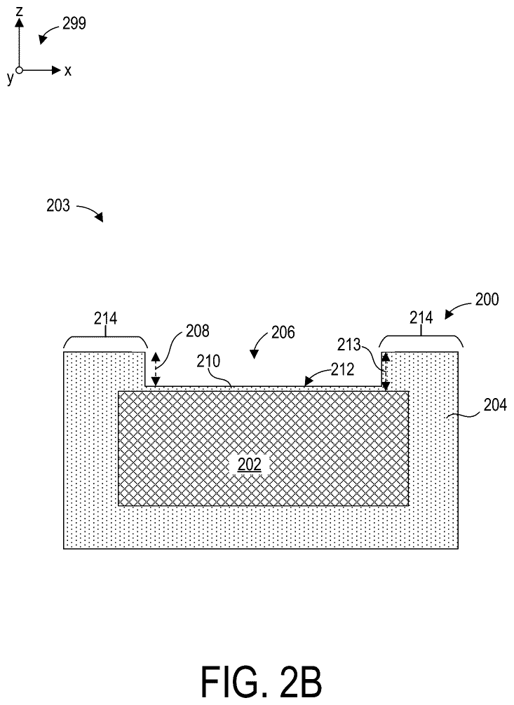

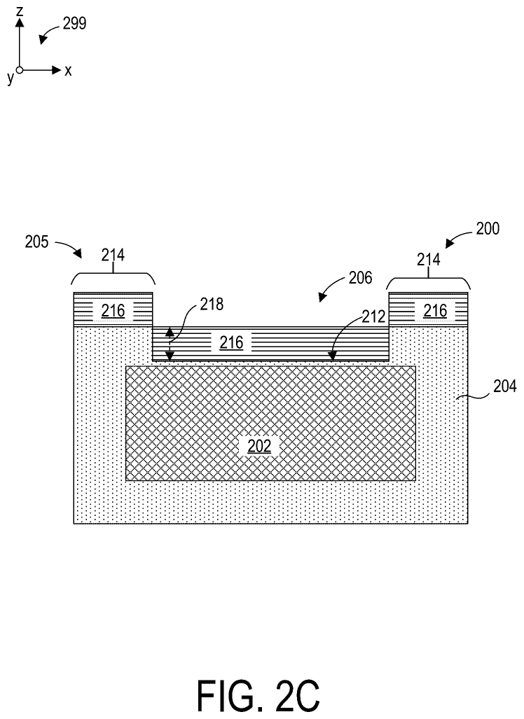

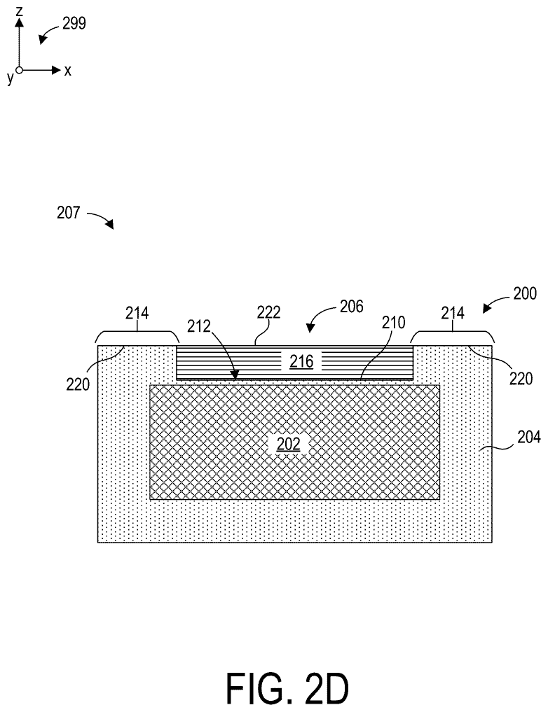

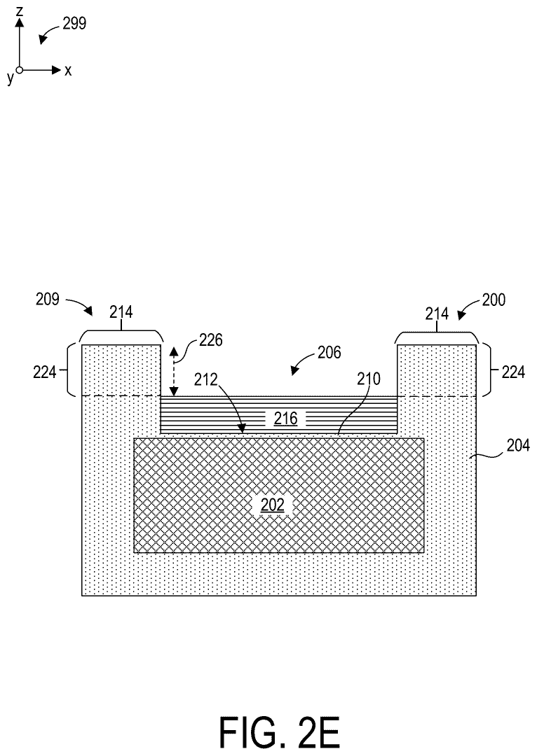

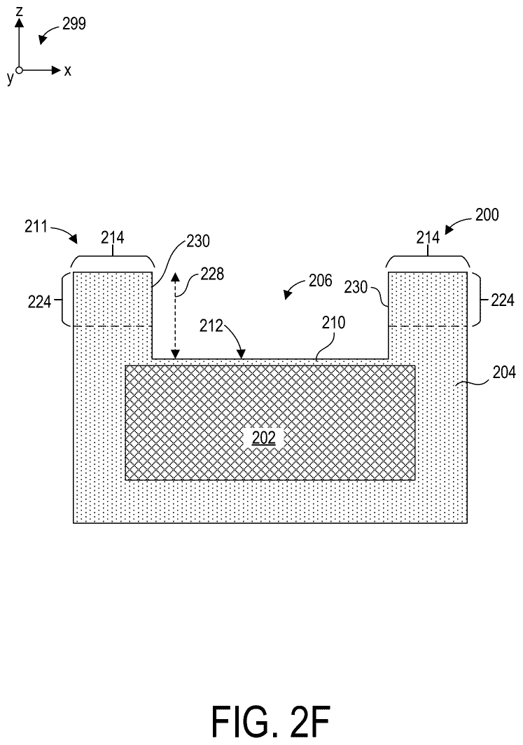

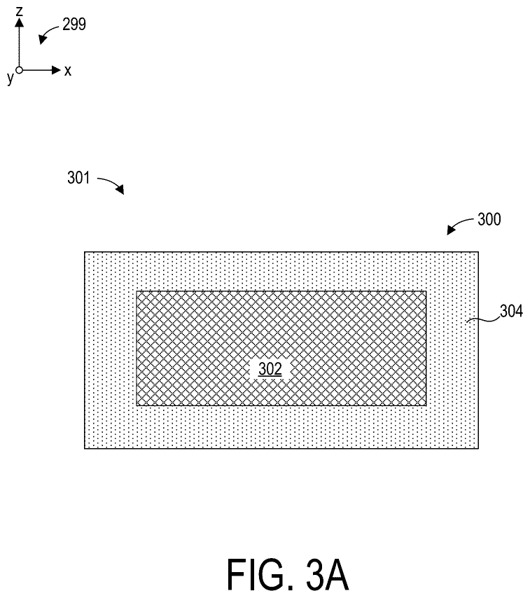

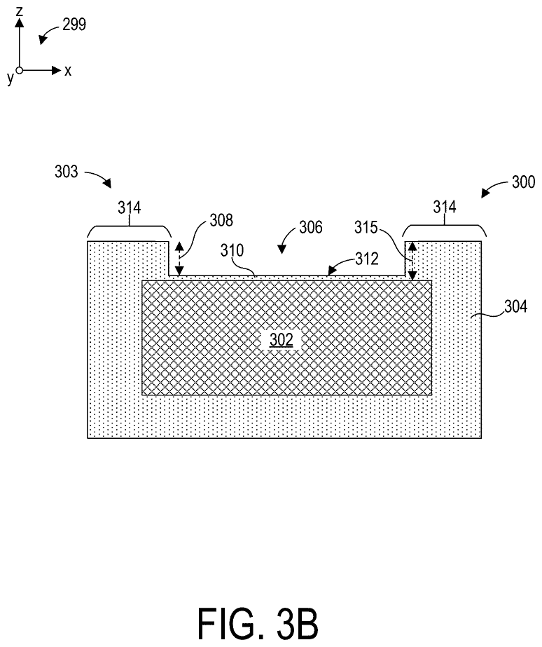

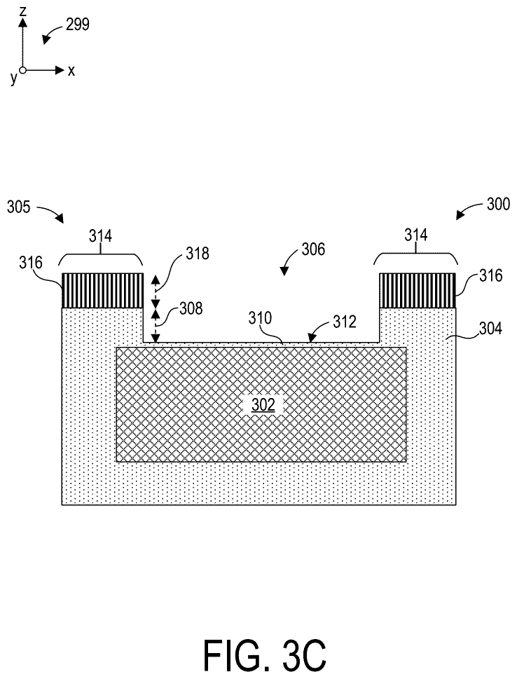

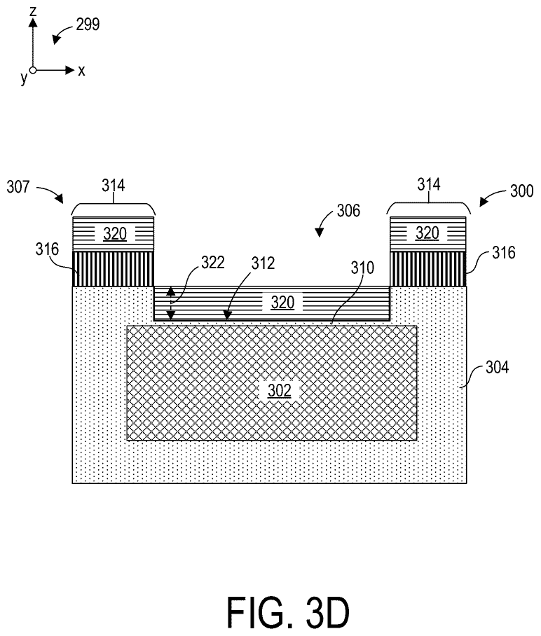

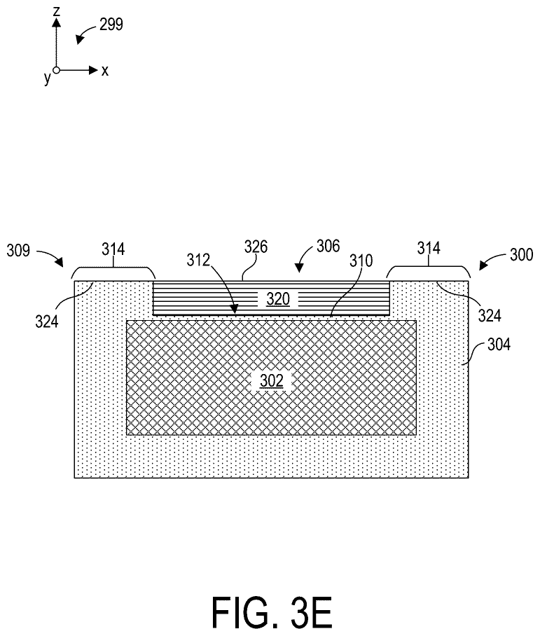

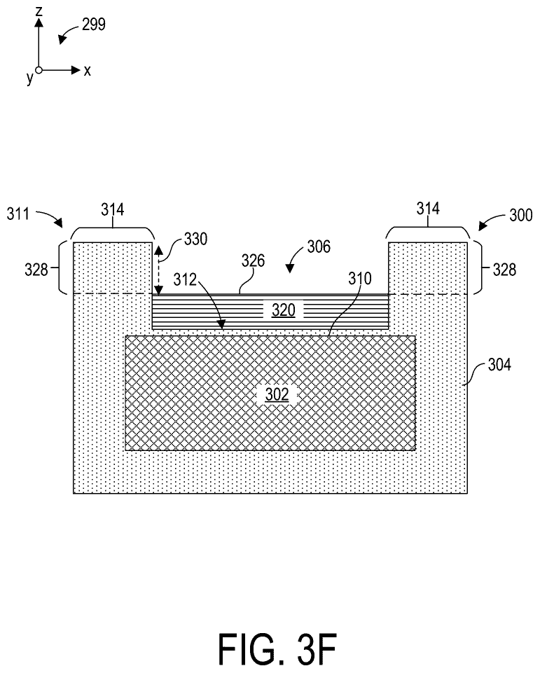

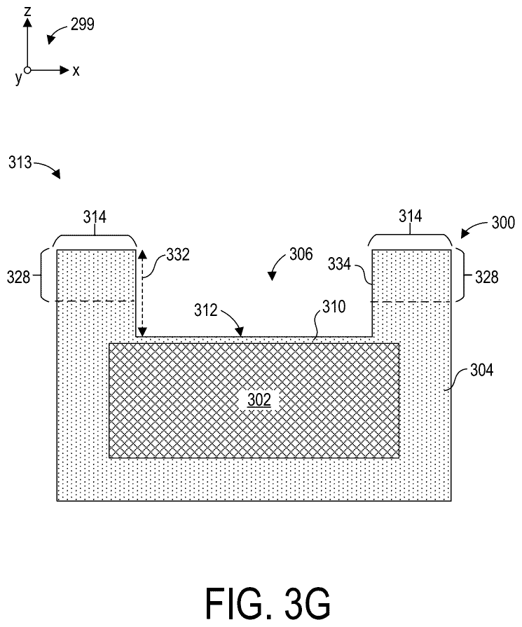

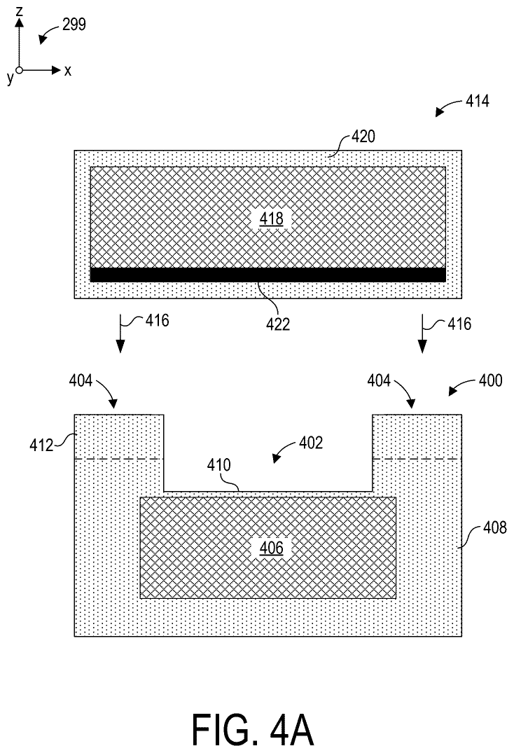

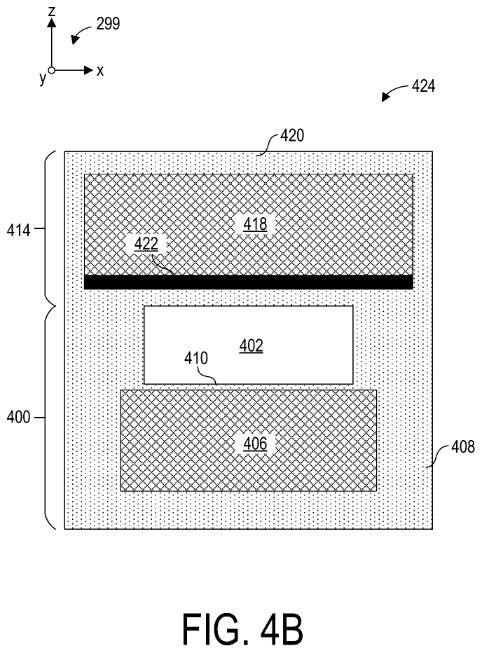

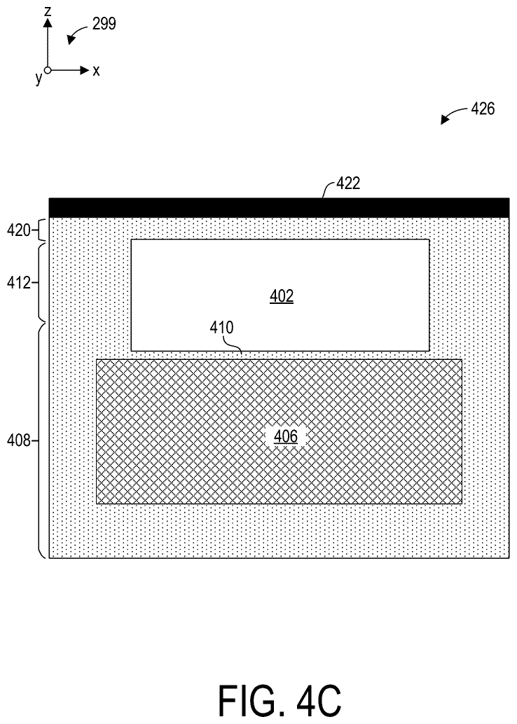

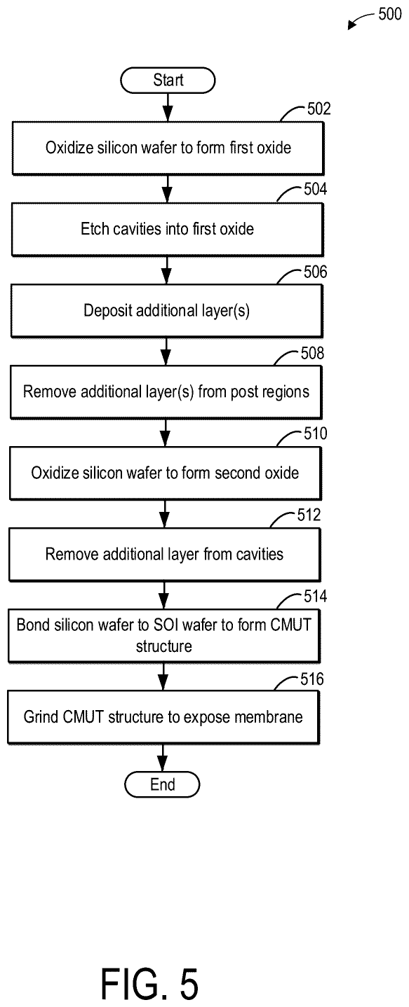

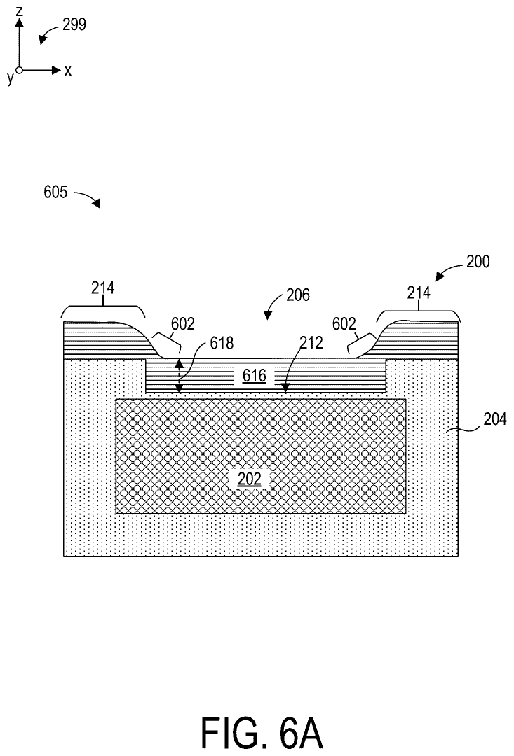

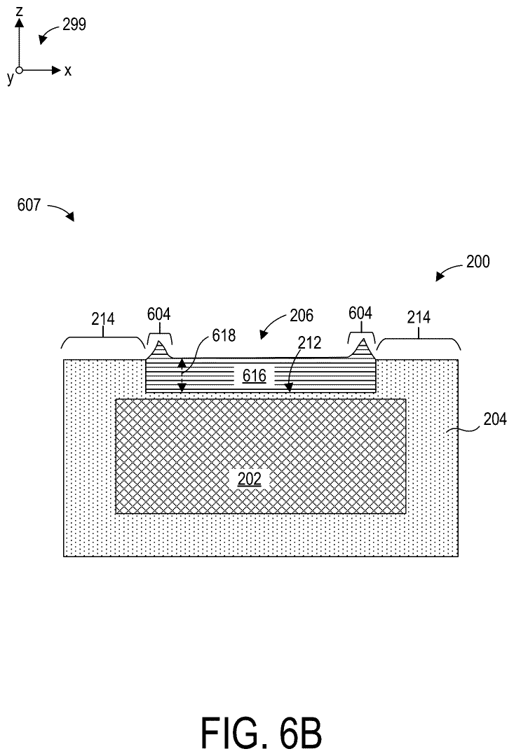

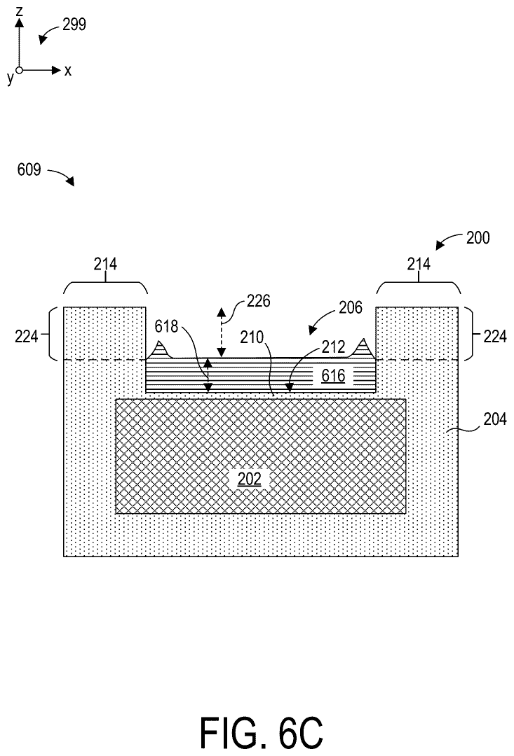

An example of a first process for treating a silicon wafer for a MEMS device, such as a CMUT, to form a substantially flat cavity floor is illustrated in FIGS. 2A-2F, as well as in FIGS. 6A-6C, and a second process for treating a silicon wafer to form a substantially flat cavity floor is depicted in FIGS. 3A-3G. Each of the first and second processes may produce a silicon wafer structure having a cavity free of bird-beak structures. The silicon wafer structure may then be coupled to a membrane to form the CMUT, as shown in FIGS. 4A-4C. An example of a method for fabricating MEMS devices via a wafer-scale approach is shown in FIG. 5.

Turning now to the first process for treating a silicon wafer for a CMUT depicted in FIGS. 2A-2F, a schematic cross-section of a silicon wafer 200 is shown having a silicon core 202 and a coating formed of a first oxide 204. The silicon wafer 200 may be representative of a single cell of a CMUT structure. The first oxide 204 (e.g., the first oxide coating) may be formed at a first step 201 of the first process, during which the silicon wafer 200 is oxidized. A set of reference axes 299 is provided, indicating a z-axis, a y-axis, and an x-axis. In one example, the z-axis may be parallel with a direction of signal propagation of a transducer probe incorporating the CMUT, the x-axis parallel with an azimuth direction, and the y-axis parallel with an elevation direction.

As shown in FIG. 2A, the first oxide 204 may be a continuous coating of silicon dioxide, produced by, for example, heating the silicon wafer 200 under oxidizing conditions. The silicon core 202 may be entirely surrounded, e.g., enclosed, by the first oxide 204. At a second step 203 depicted at FIG. 2B of the first process, a cavity 206 may be etched into an upper surface (relative to the z-axis) of the silicon wafer 200. The cavity 206 may have an initial, or first depth 208 that does not extend beyond a thickness 213 (defined along the z-axis, above the silicon core 202) of the first oxide 204. A layer 210 of the first oxide 204 may remain at a floor 212 of the cavity 206, the layer 210 continuous with the first oxide 204 at post regions 214 of the silicon wafer 200. The first depth 208 may therefore be less than the thickness 213 of the first oxide 204.

The floor 212 may be planar, e.g., substantially flat and even, and free of protruding structures or recesses. The layer 210 of the first oxide 204 remaining at the floor 212 may act as a pad oxide layer (and is hereafter referred to as the pad oxide layer 210) arranged between the silicon core 202 and a masking layer 216 deposited onto the silicon wafer 200 during a subsequent, third step 205 of the first process, as shown in FIG. 2C. Alternatively, the first process may proceed to a third step 605 of FIG. 6A, described further below.

The post regions 214 may continuously surround the cavity 206 to form a frame around the cavity 206. For example, the post regions 214 may extend continuously along the y-axis and the x-axis to form a border around the cavity 206 and may have a variety of geometries, e.g., when viewed from above along the z-axis, including circular, elliptical, square, hexagonal, etc.

The masking layer 216 is deposited over the upper surface of the silicon wafer 200 at the third step 205 (as shown in FIG. 2C) of the first process, including over the post regions 214 and a bottom (e.g., the floor 212) of the cavity 206. The masking layer 216 may be, for example, a layer of silicon nitride or some other material that is thermally stable and resistant to oxidation. A thickness 218 of the masking layer 216 may be substantially equal to the first depth 208 of the cavity 206. As such, the cavity 206 may be etched into the silicon wafer with a target depth (e.g., resulting in the first depth 208) that is equal to or less than an expected thickness of the masking layer 216. While the first depth 208 of the cavity 206 may be less than the thickness 218 of the masking layer 216, the first depth 208 of the cavity may not be greater than the thickness 218 of the masking layer 216.

As one example, the oxidation of the silicon wafer 200 during the first step 201 may be controlled to produce the first oxide 204 with a target thickness. For example, the target thickness may be a sum of a target thickness of the pad oxide layer 210 and the expected thickness of the masking layer 216. The cavity 206 may be etched to the first depth 208 according to the expected thickness of the masking layer 216, allowing the pad oxide layer 210 to remain with its target thickness.

As an example, the pad oxide layer 210 may have a thickness that is at least 10 nm. As another example, the thickness of the pad oxide layer may be 25 nm. In yet another example, the thickness of the pad oxide layer may be in a range of 10 nm to 50 nm. Thickness of the layers of the oxidized silicon wafer 200 formed via oxidation and deposited thereon may be between a few tens of nanometers up to several hundreds of nanometers.

Continuing to FIG. 2D which illustrates a fourth step 207 of the first process, the masking layer 216 is removed from the post regions 214. For example, the masking layer 216 may be removed by etching the post regions 214, while maintaining the masking layer 216 intact at the cavity 206. Upon removing the masking layer 216 from the post regions 214, upper surfaces 220 of the silicon wafer 200 at the post regions 214 may be flush with an upper surface 222 of the masking layer 216 in the cavity 206. For example, the upper surface 220 at the post regions 214 may be aligned and continuous with the upper surface 222 of the masking layer 216 along the x-y plane.

In instances where the first depth 208 (as shown in FIG. 2B) of the cavity 206 is less than the thickness 218 (as shown in FIG. 2C) of the masking layer 216, the upper surfaces 220 at the post regions 214 may not be flush with the upper surface 222 of the masking layer 216 in the cavity when the masking layer 216 is removed from the post regions 214. Instead, the upper surface 222 of the masking layer 216 may protrude higher along the z-axis than the upper surfaces 220 at the post regions 214.

A fifth step 209 of the first process is shown in FIG. 2E, which includes locally oxidizing the silicon wafer 200 at the post regions 214 (e.g., conducting a second oxidation). As an example, local oxidation of silicon (LOCOS) may be conducted to form a coating of a second oxide 224 at the post regions 214. The second oxide 224 (e.g., the second oxide coating) may increase a height of the post regions 214 along the z-axis by a field oxidation height 226 of the second oxide 224. The second oxide 224 may also be silicon dioxide and may be formed as a layer over the first oxide 204 at the post regions 214.

As shown in FIG. 2F, a sixth step 211 of the first process includes removing the masking layer 216 from the cavity 206. The masking layer 216 may be removed by, for example, etching to entirely remove the masking layer 216 and expose the pad oxide layer 210. The etching therefore does not extend into the pad oxide layer 210, thus allowing the pad oxide layer 210 to remain continuous and flat. Additionally, the floor 212 of the cavity may have a lower roughness due to a presence of the pad oxide layer 210, relative to a texture provided by the silicon core 202, if the floor 212 of the cavity 206 were instead formed of the silicon core 202.

For example, in a conventional process for forming a cavity in an oxidized silicon wafer, bird-beak structures, such as the bird-beak structure 702 shown in FIG. 7, may be formed in the cavity due to lateral diffusion of a silicon oxide produced when the silicon wafer is oxidized. Polishing of the cavity floor, such as chemical mechanical polishing (CMP), may be demanded to remove the bird-beak structures and achieve a flat cavity floor. The polishing may increase a roughness of the cavity floor and/or expose a silicon core of the silicon wafer at the cavity core, where the silicon core has rougher texture compared to the silicon oxide. In contrast, in the first process of FIGS. 2A-2F, lateral diffusion of the first or second oxides 204, 224 does not occur or may occur at a decreased rate such that bird-beak structures do not form within a time frame of the second oxidation. Further, the decreased rate of lateral diffusion may also result from different stress distribution between the masking layer 216 and the second oxide 224, as the second oxide 224 is formed at the post regions 214. The floor 212 of the cavity 206 is formed as a flat surface and remains flat throughout the first process. Polishing of the floor 212 after the masking layer 216 is removed is thereby obviated and the silicon core 202 is not exposed at the floor 212.

Upon removing the masking layer 216, the cavity 206 may have a final, second depth 228. The second depth 228 of the cavity 206 may be a sum of the first depth 208 (as shown in FIG. 2B) of the cavity 206 and the field oxidation height 226 (as shown in FIG. 2E) of the second oxide 224 at the post regions 214. As a maximum value of the first depth 208 of the cavity 206 may be the thickness 218 of the masking layer 216, a maximum value of the second depth 228 of the cavity 206 may be the sum of the thickness 218 of the masking layer 216 and the field oxidation height 226 at the post regions 214. As described above, the masking layer 216 may have a minimum thickness that is equal to the first depth 208 of the cavity 206 and may have a thickness that is greater than the first depth 208 of the cavity 206, in other examples.

The floor 212 of the cavity 206 remains substantially flat, e.g., uniformly co-planar with the x-y plane, throughout the first process. As such, a profile of the cavity 206 may include linear contours at each of the floor 212 and walls 230 of the cavity 206. As described above, by oxidizing the silicon wafer 200 before etching the cavity 206, lateral diffusion of the first oxide 204 into the cavity 206, which may otherwise result in formation of bird-beak structures, may not occur or may be substantially slower. Subsequent treatment of the cavity 206, such as etching or polishing along the floor 212 of the cavity 206, is not demanded. Further, by etching the cavity 206 to the first depth 208 such that the pad oxide layer 210 is a continuous coating over the floor 212 of the cavity 206, the pad oxide layer 210 may remain as a final layer of the floor 212 of the cavity 206, allowing the floor 212 to have a desirable smoothness. As described above, the floor 212 of the cavity 206 may have a RMS roughness value of 0.1 nm or less.

Turning now to the third step 605 of the first process shown in FIG. 6A, a masking layer 616 is deposited over the silicon wafer 200 in a similar manner as described above with reference to FIG. 2C. FIGS. 6A-6C represent alternate steps analogous to the third step 205, the fourth step 207, and the fifth step 209 of FIGS. 2C-2E, respectively and therefore components in common are similarly numbered. In the alternate third step 605, the masking layer 616 has a thickness 618 that is equal to the first depth 208 (as shown in FIG. 2B) of the cavity 206. In other examples, however, the thickness 618 of the masking layer 616 may be greater than the first depth 208 of the cavity 206. In such instances, e.g., where the masking layer thickness 618 is greater than the first depth 208 of the cavity 206, a resulting CMUT structure is the same (e.g., as shown in FIG. 2F).

In contrast to the masking layer 216 of FIGS. 2C-2E, the masking layer 616 depicted in FIG. 6A is continuous across the upper surface of the silicon wafer 200. For example, portions of the masking layer 616 at the post regions 214 merge with a portion of the masking layer 616 in the cavity 206 at merging regions 602 that are uninterrupted, e.g., without breaks or discontinuity. The merging regions 602 may be located proximate to transitions at the upper surface of the silicon wafer 200 between the post regions 214 and the cavity 206.

At a fourth step 607 of the first process depicted in FIG. 6B, the masking layer 616 is removed by etching, as described above with reference to FIG. 2D. In one example, isotropic etching may be applied. In another example, anisotropic etching may be used. The first oxide 204 is exposed at the post regions 214 while the masking layer 616 remains intact at the floor 212 of the cavity 206. In some examples, etching of the masking layer 616 at the post regions 214 may alter a geometry of the masking layer 616 at the cavity 206. For example, when the masking layer 616 is removed from the post regions 214 by wet isothermal etching, protrusions 604 may form proximate to the merging regions 602 shown in FIG. 6A. The protrusions 604 may be residual portions of the merging regions 602 after etching of the post regions 214 is conducted.

It will be noted that while the protrusions 604 formed at the alternate fourth step 607 resemble bird-beak structures, the protrusions 604 are not formed by lateral diffusion of oxide during local oxidation. Instead, the protrusions 604 are remnant structures left after mechanical processing of the post regions. As shown in FIG. 6B, the floor 212 of the cavity 206 is unaffected by the formation of the protrusions 604 and remains flat.

The silicon wafer 200 may undergo a second oxidation, e.g., local oxidation of the post regions 214, at a fifth step 609 illustrated in FIG. 6C, similar to the fifth step 209 of FIG. 2E. The height of the post regions 214 may be increased by the field oxidation height 226 of the second oxide 224, formed over the first oxide 204 at the post regions 214. The floor 212 of the cavity 206, however, is masked by the masking layer 616, and thereby not exposed to conditions of the second oxidation. The floor 212 remains substantially flat and a thickness of the pad oxide layer 210 does not change. The first process then continues to the sixth step 211 shown in FIG. 2F, where the masking layer 616 is removed from the cavity 206.

The second process for treating a silicon wafer for a CMUT is illustrated in FIGS. 3A-3G. The second process may include steps analogous to the steps of the first process of FIGS. 2A-2F but includes an additional step of depositing a sacrificial layer onto a silicon wafer 300. The silicon wafer 300 is depicted as a schematic cross-section and may represent a single cell of a CMUT structure, which may include any number of the single cell. As shown FIG. 3A, at a first step 301 of the second process, the silicon wafer 300 is oxidized to form a coating of a first oxide 304 that continuously surrounds a silicon core 302 of the silicon wafer 300. As described above, the first oxide 304 may be silicon dioxide. More specifically, the first oxide may be a thermally grown oxide that is formed via a dry or wet process and not by layer deposition.

During a second step 303, as shown in FIG. 3B, of the second process, a cavity 306 may be etched into the first oxide 304. As described above with reference to the second step 203 of FIG. 2B, the cavity 306 may have a first depth 308 that is equal to or less than an expected thickness of a masking layer that is deposited onto the silicon wafer 200 in a subsequent step of the second process.

A pad oxide layer 310, formed of a layer of the first oxide 304, may remain as a floor 312 of the cavity 306 after the cavity 306 is etched. The first depth 308 of the cavity 306 may therefore be less than a thickness 315 of the first oxide 304. As described above with reference to the first process of FIGS. 2A-2F and/or FIGS. 6A-6C, the floor 312 may be substantially flat and free of protruding or receding structures. The pad oxide layer 310 may be continuous with the first oxide 304 at post regions 314 of the silicon wafer 300, the post regions 314 continuously surrounding the cavity 306.

At a third step 305, as illustrated in FIG. 3C, of the second process, a sacrificial layer 316 is deposited onto the post regions 314. In one example, the sacrificial layer 316 may be a polymer, such as a photoresist layer deposited onto the post regions 314 by photolithography. In other examples, the sacrificial layer 316 may be any other type of material able to withstand processing conditions of the second process, particularly during masking layer deposition and a second oxidation (e.g., as shown in FIG. 3F).

The sacrificial layer 316 may have a thickness 318 that is selected according to an anticipated deposition of a masking layer (e.g., a masking layer 320 shown in FIG. 3D) over the silicon wafer 300. For example, the thickness 318 may be selected to ensure that the masking layer at the floor 312 of the cavity 306 is not continuous with the masking layer at the post regions 314 once the masking layer is deposited, in contrast to the continuity of the masking layer 616 shown in FIG. 6A. In other words, the thickness 318 of the sacrificial layer disrupts a continuity of the masking layer between the cavity 306 and the post regions 314. As an example, the thickness 318 of the sacrificial layer 316 may be increased for a given anticipated masking layer thickness when the first depth 308 of the cavity 306 is less than the anticipated masking layer thickness compared to when the first depth 308 of the cavity 306 is equal to the anticipated masking layer thickness. By maintaining the masking layer discontinuous between the floor 312 and the post regions 314, subsequent removal of the masking layer at the post regions 314 may be achieved without mechanical techniques, as described further below.

A material of the sacrificial layer 316 may be selected based on chemical properties of the sacrificial layer material that differ from chemical properties of the silicon wafer 300. For example, the sacrificial layer material may be readily dissolved, e.g., chemically etched, upon exposure to a chemical that does not dissolve any other portions of the silicon wafer 300. Further, the sacrificial layer 316 may not adhere or bond to the first oxide 304 at the post regions 314.

As shown in FIG. 3D, the masking layer 320 is deposited onto the silicon wafer 200 at a fourth step 307 of the second process. The masking layer 320 may form a coating over the floor 312 of the cavity 306 and at the post regions 314, on top of the sacrificial layer 316. The masking layer 320 may have a thickness 322 that is equal to or greater than the first depth 308 (as shown in FIG. 3B) of the cavity 306.

As described above, a presence of the sacrificial layer 316 at the post regions 314, deposited prior to deposition of the masking layer, disrupts a continuity of the masking layer 320 across the silicon wafer 300. For example, a portion of the masking layer 320 at the floor 312 of the cavity 306 is separate and spaced away from portions of the masking layer 320 at the post regions 314 by the thickness 318 (as shown in FIG. 3C) of the sacrificial layer 316.

At a fifth step 309 of the second process, depicted in FIG. 3E, the sacrificial layer 316 and the masking layer 320 are removed from the post regions 314. In one example, the layers may be removed by a lift-off process, which is enabled by a presence of the sacrificial layer 316 as well as a lack of adherence or bonding of the sacrificial layer 316 to the first oxide 304. The lift-off process may include removing the sacrificial layer 316 by chemical etching. As the sacrificial layer 316 is chemically etched, the masking layer 320 at the post regions 314 is concomitantly detached therefrom. The masking layer 320 at the floor 312 of the cavity 306, however, remains intact. As a result of the lift-off process, final treatment of upper surfaces 324 of the post regions 314, such as CMP, is not demanded to achieve flat surfaces at the post regions 314 subsequent to removal of the sacrificial layer 316. A texture of the upper surfaces 324 of the post regions 314 may be maintained within a target range of roughness (e.g., a low degree of roughness, such as equal to or less than a RMS roughness value of 0.1 nm) by precluding etching of the upper surfaces 324 after the sacrificial layer 316 is removed. An adherence of a membrane to the post regions 314 of the silicon wafer 300 may be more robust by maintaining the upper surfaces 324 of the post regions 314 with a desirable smoothness, even after a second oxidation of the silicon wafer 200.

As shown in FIG. 3E, upon removing the sacrificial layer 316 and the masking layer 320 from the post regions, the upper surfaces 324 of the post regions 314 may be flush, e.g., even along the x-y plane, with an upper surface 326 of the masking layer 320 in the cavity 306 when the thickness 322 of the masking layer 320 (as shown in FIG. 3D) is equal to the first depth 308 (as shown in FIG. 3B) of the cavity 306. In examples, however, where the first depth 308 of the cavity 306 is less than the thickness 322 of the masking layer 320, the upper surfaces 324 of the post regions 314 may not be flush with the upper surface 326 of the masking layer 320. In such examples, the upper surface 326 of the masking layer 320 may protrude higher than the upper surfaces 324 of the post regions 314.

As shown in FIG. 3F, the silicon wafer is locally oxidized, e.g., via LOCOS, at a sixth step 311 of the second process. As described above, with reference to the fifth step 209 of FIG. 2E, local oxidation of the silicon wafer may result in formation of a coating of a second oxide 328 over the first oxide 304 at the post regions 314. The formation of the second oxide 328 may increase a height of the post regions 314 by a field oxidation height 330 of the second oxide 328. The second oxide 328 may also be silicon dioxide.

At a final, seventh step 313 of the second process, illustrated in FIG. 3G, the masking layer 320 is removed from the cavity 306. For example, the masking layer 320 may be removed from the cavity 306 by etching (e.g., mechanically etching) the masking layer 320. After removal of the masking layer 320, the cavity 306 may have a final, second depth 332 that is equal to a sum of the first depth 308 (as shown in FIG. 3B) of the cavity 306 and the field oxidation height 330 (as shown in FIG. 3F) of the second oxide 328. When the first depth 308 of the cavity 306 is equal to the thickness 322 (as shown in FIG. 3D) of the masking layer 320, the second depth 332 of the cavity 306 may also be a sum of the thickness 322 of the masking layer 320 and the field oxidation height 330. A final profile of the cavity 306 may include the uniformly and substantially flat floor 312 with linear walls 334 of the cavity 306 extending perpendicular to the floor 312.

As described above with respect to the first process of FIGS. 2A-2F, the second process of FIGS. 3A-3G also results in formation of a MEMS device cavity with a substantially flat floor that is continuously coated with a first oxide. The first oxide is formed before the cavity is etched into the silicon wafer, allowing the floor of the cavity to remain planar and free of protruding and receding structures after processing is complete. The cavity floor may have a desirably low roughness measurement, as may the upper surfaces of the post regions. Although the second process may include an additional processing step compared to the first process, the use of a sacrificial layer in the second process may increase a reliability of a resulting MEMS device by increasing a bonding strength between the upper surfaces of the post regions and a membrane (e.g., a membrane for a CMUT). For example, the bonding strength may be increased by upper surfaces of the post regions that are more smooth relative to upper surfaces of post regions that have been treated by polishing.

Furthermore, the second process may provide preclude formation of protrusions at the masking layer in the cavity, such as the protrusions 604 of FIG. 6B, after removal of the masking layer from the post regions. As described above with respect to FIG. 6B, the protrusions may be remnant portions of merging regions (e.g., the merging regions 602 of FIG. 6A) of the masking layer when the masking layer is continuous across the post regions and the cavity. Although the presence of the protrusions at the masking layer in the cavity does not affect the pad oxide layer under the masking layer, in instances where the masking layer is left in the cavity for a period of time, the second process obviates a demand to remove the protrusions from the masking layer by etching.

A silicon wafer treated by either of the first process or the second process may be incorporated into a MEMS device, such as a CMUT, by coupling a membrane to the silicon wafer. An example of a process for forming the CMUT is illustrated in FIGS. 4A-4C. For example, as shown in FIG. 4A, a treated silicon wafer 400 may be configured similarly to the silicon wafer 200 of FIG. 2F or the silicon wafer 300 of FIG. 3G, having a cavity 402 framed by post regions 404. As described previously, the treated silicon wafer 400 is depicted as a schematic cross-section and represents a single cell of a CMUT structure. The treated silicon wafer 400 may have a silicon core 406 continuously surrounded by a first oxide 408, which also forms a floor 410 of the cavity. The post regions 404 may also include a second oxide 412, formed on top of the first oxide 408 (relative to the z-axis) and continuous with the first oxide 408 such that the second oxide 412 may be distinguishable from the first oxide 408 but not removed or separated from the first oxide 408.

An oxidized Silicon on Insulator (SOI) wafer 414 may be coupled to the treated silicon wafer 400 as indicated by arrows 416. The oxidized SOI wafer 414 may have a silicon core 418, an oxide layer 420 and a membrane 422. The oxide layer 420 may be formed by oxidizing the silicon core 418, along with the membrane 422 coupled thereto, which may result in the oxide layer 420 forming a continuous outer layer of the SOI wafer 414. The oxide layer 420 may have a thickness in a range of a few tens of nanometers up to a few hundred nanometers. The oxide layer 420 may be formed to have a thickness that is defined by a target insulation layer thickness requirement of structure of the CMUT, which may in turn, be dependent on demands of a specific application for the CMUT. Further, the oxide layer thickness may be optimized according to a configuration of the CMUT structure as well as a minimum thickness required to accommodate a bonding process of the SOI wafer to the treated silicon wafer 400, as elaborated below.

The membrane 422 may be formed of a layer of the same material as the silicon core and/or stacks of silicon carbide, silicon, silicon nitride, polysilicon, etc. arranged on the layer, along with a metal, such as chromium, gold, and/or aluminum, to form an electrode that is incorporated into the membrane 422. The SOI wafer 414, upon coupling to the silicon wafer 400 may be oriented relative to the silicon wafer 400 with the membrane 422 proximate to the silicon wafer 400. The SOI wafer 414 may be attached to the silicon wafer 400 to form a CMUT structure 424, as shown in FIG. 4B, by fusion bonding, as one example, although other bonding procedures may be used.

The CMUT structure 424 may be a single, continuous structure with the oxide layer 420 of the SOI wafer 414 bonded to the second oxide 412 of the silicon wafer 400 at the post regions 404 such that the oxide layer 420 is fixedly coupled to the second oxide 412. As a result, the cavity 402 may be entirely enclosed by and sealed within a combination of the oxide layer 420 of the SOI wafer 414, and the first and second oxides 408, 412 of the cavity 402. In one example, during bonding of the SOI wafer 414 to the silicon wafer 400, the cavity 402 may be vacuum sealed, e.g., sealed at a pressure lower than ambient pressure, within the CMUT structure 424 to form a vacuum chamber. In other examples, the cavity 402 may be sealed with fluid, such as air, therein.

The CMUT structure 424 may undergo grinding to provide a CMUT 426, as depicted in FIG. 4C, the CMUT 426 formed of one or more of the CMUT structure 424. For example, a portion of the CMUT structure 424 formed from the SOI wafer 414 may be ground from a top of the CMUT structure 424 downwards, removing a portion of the SOI wafer 414 above the membrane 422. The membrane 422 may thereby be an uppermost layer of the CMUT 426, with an upper surface of the membrane 422 forming an upper face of the CMUT 426. The oxide layer 420 of the SOI wafer 414 may form an underside of the membrane 422 and vibrate in unison with the membrane 422 when the CMUT 426 generates or receives ultrasonic signals.

The CMUT 426 may therefore be fabricated with the cavity 402 separating the membrane 422 from the treated silicon wafer 400. The floor 410 of the cavity 402 may be substantially flat and free of bird-beak structures which may otherwise contact the underside of the membrane 422 and adversely affect a signal transmitted or received by the CMUT 426, particularly during operation of the CMUT 426 in a collapse-mode. As described above, the floor 410 of the cavity 402 may have a desirable smoothness that minimizes a likelihood of electrical charging within the cavity 402.

The treatment of the silicon wafer illustrated in FIGS. 2A-2F, 6A-6C, and 3A-3G, and the manufacturing of the CMUT by coupling a membrane to the treated silicon wafer, as shown in FIGS. 4A-4C, may be used in a wafer-scale fabrication process of MEMS devices. The wafer-scale fabrication process may enable high throughout production of the MEMS devices at relatively low cost. An example of a method 500 for fabricating a MEMS device is shown is FIG. 5, which may be a wafer-scale manufacturing process. The MEMS device may be a CMUT, such as the CMUT 426 of FIG. 4C, and may be formed from a silicon wafer treated by either of the processes shown in FIGS. 2A-2F and 3A-3G. The method 500 may be executed by an automated system, including suitable instruments and machines, by an operator, or by a combination thereof, at a production facility.

At 502, the method includes oxidizing the silicon wafer to form a first oxide that entirely coats a silicon core of the wafer. The oxidation of the silicon wafer may be controlled to achieve a target thickness of the first oxide. For example, the first oxide thickness may be equal to a sum of a desired pad oxide layer thickness and an expected masking layer thickness.

Cavities are etched into the oxidized silicon wafer at 504. As an example, the cavities may be etched from an upper surface of the silicon wafer, into the thickness of the first oxide. In one example, the cavities may be etched with a depth that is equal to the expected thickness of the masking layer, allowing the desired pad oxide layer thickness to remain, the pad oxide layer formed of a remaining layer of the first oxide at floors of the cavities. In another example, the cavities may be etched with a depth that is less than the expected thickness of the masking layer with desired pad oxide layer thickness similarly maintained after etching. Upon completion of the etching, the floors of the cavities may be substantially flat and smooth. Further, the pad oxide layer may be continuous with the first oxide along walls of the cavities and with the first oxide at post regions of the silicon wafer. The post regions may be regions of elevated height relative to the cavity floors that remain after the cavities are etched.

At 506, the method includes depositing one or more additional layers onto the silicon wafer. For example, the masking layer may be deposited over the post regions and the floors of the cavities. In another example, the one or more additional layers may include a sacrificial layer that is deposited at the post regions by a technique that allows deposition of a material onto the silicon wafer according to a predetermined pattern, such as photolithography. The sacrificial layer may be located only at the post regions after deposition, and may be applied before the masking layer is deposited.

The additional layer(s) are removed from the post regions of the silicon wafer at 508. In examples where only the masking layer is deposited onto the silicon wafer, the masking layer may be etched from the post regions. After removal of the masking layer, the post regions may be treated by polishing, such as CMP, to produce planar (e.g., flat), smooth upper surfaces of the post regions.

In examples where the additional layers include the sacrificial layer at the post regions as well as the masking layer, the sacrificial layer and the masking layer may be removed from the post regions by lift-off. For example, the sacrificial layer may be removed by chemical etching, which also removes the masking layer from the post regions.

At 510, the silicon wafer is oxidized to form a second oxide. For example, LOCOS may be conducted, which may drive formation of the second oxide over exposed regions of the first oxide, such as at the post regions where the additional layer(s) have been removed. At the cavities, however, the masking layer shields the cavity floors from oxidizing conditions that the silicon wafer is subjected to, inhibiting oxidation of the first oxide at the cavity floors. Oxidation of the first oxide at the post regions causes the second oxide to form on top of the first oxide, which may increase a height of the post regions. For example, the height of the post regions may increase by a field oxidation height of the second oxide.

At 512, the additional layer, e.g., the masking layer, is removed from the floors of the cavities. In one example, the masking layer may be removed by etching the masking layer along the cavity floors. The etching may be performed such that the pad oxide layer (e.g., the layer of the first oxide remaining as a coating over the cavity floors) remains intact, continuous, and planar after the masking layer is removed. Further, after removal of the masking layer, the pad oxide layer may have a relatively smooth surface, with a RMS roughness measurement of 0.1 nm or less.

A SOI wafer is bonded to the silicon wafer at 514 to form a CMUT structure. The SOI wafer may include a membrane coupled to a silicon core of the SOI wafer with an oxide layer forming a continuous outer layer of the SOI wafer. For example, to form the SOI wafer, a silicon substrate may be coupled to the membrane and then the silicon substrate and membrane may undergo an oxidation process. The oxide layer is formed as a result of the oxidation process.

The SOI wafer may be coupled to the silicon wafer by fusion bonding, as one example. By bonding the SOI wafer to the silicon wafer with the membrane of the SOI wafer arranged proximate to the silicon wafer, the oxide layer arranged along an outer face of the membrane, e.g., on a face opposite of the silicon core of the SOI wafer, may be adhered to the second oxide at the post regions of the silicon wafer. The oxide layer may also form an underside of the membrane, vibrating in unison with the membrane and forming ceilings of the cavities of the silicon wafer.

Upon coupling the SOI wafer to the silicon wafer by, for example, fusion bonding, the cavities may be sealed within the CMUT structure such that air (or fluid) outside of the CMUT structure does not exchange with air (or fluid) inside the cavities. Further, the bonding of the SOI wafer to the silicon wafer may be conducted under low pressure, e.g., vacuum conditions, such that the cavities are sealed under vacuum and remain at low pressure (e.g., lower than ambient pressure) after bonding is complete. Inner surfaces of the cavities are formed of a combination of the first and second oxides of the silicon wafer and the oxide layer of the SOI wafer.

At 516, grinding is applied to the CMUT structure to remove a portion of the SOI wafer above the membrane. A CMUT is provided when the grinding is complete. For example, the oxide layer and the silicon core of the SOI wafer may be ground to expose a surface of the membrane opposite of the remaining oxide layer that forms the ceilings of the cavities. The membrane forms a top of the CMUT structure, located above the cavities with the remaining oxide layer of the SOI wafer arranged between the membrane and the cavities, as well as between the membrane and the second oxide of the post regions of the silicon wafer. The CMUT structure may be a single continuous structure incorporating the treated silicon wafer and ground SOI wafer and including the cavities with substantially flat floors that are continuously coated with the pad oxide layer of the silicon wafer. Further, the cavity floors may be uniformly spaced away from the membrane. The silicon core of the silicon wafer is not exposed at the cavity floors due to the continuous coating of the cavity floors with the pad oxide layer, which is continuous with the first oxide at the walls of the cavities.

In this way, a MEMS device may be fabricated via a wafer-scale approach that enables control over a profile of cavities formed therein. The processes and techniques described above may allow the MEMS device to have a cavity profile with a target cavity depth and with substantially and uniformly flat floors that are free of structures that may otherwise interfere with vibration of a membrane of the MEMS device during operation. In addition, the cavity floors may have a desirable smoothness that may reduce a likelihood of electrical charging. The cavity floor profile may be obtained according to desired dimensions and geometry via a streamlined process that includes fewer etching steps that conventional processes and precludes polishing of the cavity floors to achieve a flat floor, thereby maintaining a roughness of the cavity floors within a target range. Further, in some examples, polishing of the post regions to enable secure bonding of the membrane to the post regions may also be obviated when a sacrificial layer is applied to the post regions before deposition of a masking layer, allowing the sacrificial layer to be removed by a lift-off process. The systems and methods described herein for treating a silicon wafer of a MEMS device may provide low cost, effective strategies for optimizing a performance and longevity of the MEMS device.

FIGS. 2A-4C, and 6A-6C show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

The disclosure also provides support for a microelectromechanical systems (MEMS) device, comprising: a silicon wafer with a first oxide coating, a cavity in the first oxide coating having a substantially flat floor formed of a layer of the first oxide coating that is continuous with the first oxide coating at post regions of the MEMS device, the cavity etched to a first depth prior to deposition of a masking layer, and a membrane coupled to the silicon wafer and spaced away from the substantially flat floor of the cavity by a second depth of the cavity, wherein the second depth is a sum of the first depth and a height of a second oxide coating formed at the post regions of the MEMS device after the masking layer is removed from the post regions. In a first example of the system, the MEMS device is a capacitive micromachined ultrasonic transducer (CMUT). In a second example of the system, optionally including the first example, the first oxide coating continuously coats surfaces of the silicon wafer, including at the substantially flat floor of the cavity. In a third example of the system, optionally including one or both of the first and second examples, the first depth is equal to or less than a thickness of the masking layer. In a fourth example of the system, optionally including one or more or each of the first through third examples, neither the first depth nor the second depth of the cavity extends into the silicon wafer. In a fifth example of the system, optionally including one or more or each of the first through fourth examples, the second depth of the cavity is equal to a thickness of the masking layer plus a field oxidation height at the post regions. In a sixth example of the system, optionally including one or more or each of the first through fifth examples, surfaces of the post regions are not etched. In a seventh example of the system, optionally including one or more or each of the first through sixth examples, a roughness of the substantially flat floor, the roughness determined by root mean square, is equal to or less than 0.1 nm.

The disclosure also provides support for a method for forming a microelectromechanical systems (MEMS) device, comprising: etching a cavity into a first oxide coating of a substrate, depositing one or more additional layers onto the substrate, the one or more additional layers including a masking layer deposited onto a bottom of the cavity and post regions around the cavity, removing the one or more additional layers from the post regions to form a flush surface between the post regions and a portion of the masking layer remaining on the bottom of the cavity, locally oxidizing the post regions to form a second oxide coating over the first oxide coating, and removing the portion of the masking layer from the cavity to produce a cavity profile with a substantially flat floor. In a first example of the method, the cavity is etched to a depth equal to or less than a thickness of the masking layer. In a second example of the method, optionally including the first example, the one or more additional layers further includes a sacrificial layer, the sacrificial layer deposited at the post regions before the masking layer is deposited, and wherein the sacrificial layer has thickness that disrupts a continuity of the masking layer between the cavity and the post regions. In a third example of the method, optionally including one or both of the first and second examples, the sacrificial layer is a photoresist layer deposited using photolithography. In a fourth example of the method, optionally including one or more or each of the first through third examples, the one or more additional layers are removed from the post regions by chemical etching. In a fifth example of the method, optionally including one or more or each of the first through fourth examples, removing the one or more additional layers from the post regions includes etching the one or more additional layers from the post regions. In a sixth example of the method, optionally including one or more or each of the first through fifth examples after the portion of the masking layer is removed from the cavity, a layer of the first oxide coating remains at the substantially flat floor of the cavity, the layer of the first oxide coating continuous with the first oxide coating at the post regions. In a seventh example of the method, optionally including one or more or each of the first through sixth examples, the method further comprises: oxidizing the substrate to form the first oxide coating before etching the cavity, and wherein etching the cavity includes not etching the cavity deeper than a thickness of the first oxide coating to provide a pad oxide layer at the substantially flat floor of the cavity.

The disclosure also provides support for a capacitive micromachined ultrasonic transducer (CMUT), comprising: a substrate having cavities with substantially flat floors continuously coated with an oxide, the cavities having final depths equal to a thickness of a masking layer plus a field oxidation height at post regions of the substrate. In a first example of the system, the substantially flat floors are free of protrusions and indentations. In a second example of the system, optionally including the first example, a thickness of the oxide at the substantially flat floors is greater than or equal to 10 nm. In a third example of the system, optionally including one or both of the first and second examples, the substrate is continuously coated with the oxide by oxidizing the substrate before the cavities are etched into the oxide, and wherein the cavities are etched only into the oxide and not into a silicon core of the substrate.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A microelectromechanical systems (MEMS) device, comprising:
a silicon wafer with a first oxide coating;
a cavity in the first oxide coating having a substantially flat floor formed of a layer of the first oxide coating that is continuous with the first oxide coating at post regions of the MEMS device, the cavity etched to a first depth prior to deposition of a masking layer; and
a membrane coupled to the silicon wafer and spaced away from the substantially flat floor of the cavity by a second depth of the cavity, wherein the second depth is a sum of the first depth and a height of a second oxide coating formed at the post regions of the MEMS device after the masking layer is removed from the post regions.

2. The MEMS device of claim 1, wherein the MEMS device is a capacitive micromachined ultrasonic transducer (CMUT).

3. The MEMS device of claim 1, wherein the first oxide coating continuously coats surfaces of the silicon wafer, including at the substantially flat floor of the cavity.

4. The MEMS device of claim 1, wherein the first depth is equal to or less than a thickness of the masking layer.

5. The MEMS device of claim 1, wherein neither the first depth nor the second depth of the cavity extends into the silicon wafer.

6. The MEMS device of claim 1, wherein the second depth of the cavity is equal to a thickness of the masking layer plus a field oxidation height at the post regions.

7. The MEMS device of claim 1, wherein surfaces of the post regions are not etched.

8. The MEMS device of claim 1, wherein a roughness of the substantially flat floor, the roughness determined by root mean square, is equal to or less than 0.1 nm.

9. A method for forming a microelectromechanical systems (MEMS) device, comprising:
etching a cavity into a first oxide coating of a substrate;
depositing one or more additional layers onto the substrate, the one or more additional layers including a masking layer deposited onto a bottom of the cavity and post regions around the cavity;
removing the one or more additional layers from the post regions to form a flush surface between the post regions and a portion of the masking layer remaining on the bottom of the cavity;
locally oxidizing the post regions to form a second oxide coating over the first oxide coating; and
removing the portion of the masking layer from the cavity to produce a cavity profile with a substantially flat floor.

10. The method of claim 9, wherein the cavity is etched to a depth equal to or less than a thickness of the masking layer.

11. The method of claim 9, wherein the one or more additional layers further includes a sacrificial layer, the sacrificial layer is deposited at the post regions before the masking layer is deposited, and wherein the sacrificial layer has thickness that disrupts a continuity of the masking layer between the cavity and the post regions.

12. The method of claim 11, wherein the sacrificial layer is a photoresist layer deposited using photolithography.

13. The method of claim 9, wherein the one or more additional layers are removed from the post regions by chemical etching.

14. The method of claim 9, wherein removing the one or more additional layers from the post regions includes etching the one or more additional layers from the post regions.

15. The method of claim 9, wherein, after the portion of the masking layer is removed from the cavity, a layer of the first oxide coating remains at the substantially flat floor of the cavity, the layer of the first oxide coating continuous with the first oxide coating at the post regions.

16. The method of claim 9, further comprising oxidizing the substrate to form the first oxide coating before etching the cavity, and wherein etching the cavity includes not etching the cavity deeper than a thickness of the first oxide coating to provide a pad oxide layer at the substantially flat floor of the cavity.

17. A capacitive micromachined ultrasonic transducer (CMUT), comprising:
a substrate having cavities with substantially flat floors continuously coated with an oxide, the cavities having final depths equal to a thickness of a masking layer plus a field oxidation height at post regions of the substrate.

18. The CMUT of claim 17, wherein the substantially flat floors are free of protrusions and indentations.

19. The CMUT of claim 17, wherein a thickness of the oxide at the substantially flat floors is greater than or equal to 10 nm.

20. The CMUT of claim 17, wherein the substrate is continuously coated with the oxide by oxidizing the substrate before the cavities are etched into the oxide, and wherein the cavities are etched only into the oxide and not into a silicon core of the substrate.
